(19) United States
(12) Patent Application Publication
(10) Pub. No.: US 2026/0328767 A1
(43) Pub. Date: Oct. 22, 2026

(54) DIAGNOSTIC TESTING APPARATUS AND SYSTEM

(71) Applicant: RAPID VIRAL DETECTION SYSTEMS LLC, Orlando, FL (US)

(72) Inventors: Ramesh Babu Vallabhaneni, Orlando, FL (US); Ronald H. Leventhal, Orlando, FL (US); William S. Kenefick, Orlando, FL (US)

(73) Assignee: RAPID VIRAL DETECTION SYSTEMS LLC, Orlando, FL (US)

(21) Appl. No.: 19/105,730

(22) PCT Filed: Aug. 31, 2023
(86) PCT No.: PCT/US2023/031648

§ 371 Date: Feb. 21, 2025

Related U.S. Application Data
(60) Provisional application No. 63/402,746, filed on Aug. 31, 2022.

(51) Int. Cl. G01N 33/569; G01N 27/622; G01N 33/543

(52) U.S. Cl. CPC G01N 33/56983; G01N 27/622; G01N 33/54366; G01N 2333/165

(57) ABSTRACT

A method and an apparatus utilizing targeted ion mobility spectrometry for the detection of the SARS-CoV-2 virus and its variants, by measuring the quantity of free polyamines including putrescine, spermidine, and spermine in a sublingual saliva sample. Other embodiments are capable of providing instant, cost effective, POC testing and test results for other viral and bacterial infections including influenza, acute and chronic respiratory conditions, certain forms of inflammation, and the detection of certain abnormal cells in human subjects.

### CROSS-REFERENCE TO RELATED APPLICATIONS

This application claims priority to U.S. Provisional Application Ser. No. 63/402,746, filed Aug. 31, 2022, which is incorporated by reference in its entirety.

### TECHNICAL FIELD

This disclosure generally relates to methods and systems utilizing targeted ion mobility spectrometry for the detection of the SARS-CoV-2 virus that causes COVID-19 and for other viral and bacterial infections.

### BACKGROUND

The current art is generally comprised of diagnostic testing systems utilizing serum samples, nasal samples, or oropharyngeal samples that often impose discomfort on test subjects and typically necessitate the use of trained medical professionals for portions of the testing. The current art also typically fails to produce immediate and highly accurate results, typically requiring the utilization of costly laboratory resources that extend test completion times and the reporting of results for as much as several days.

### SUMMARY

The systems and methods herein may allow highly accurate diagnostic testing for certain viral or bacterial infections to be conducted by laypersons using non-invasively collected test samples in order to provide consistently accurate and immediate results.

In one embodiment, testing provides for the qualitative detection of SARS-CoV-2 that causes COVID-19. The systems and methods may utilize ion mobility spectrometry (IMS), isolating and detecting ionized molecules of specified volatile organic chemicals, specifically certain polyamines derived from the precursor ornithine. The systems and methods may incorporate a transportable architecture similar in form factor and general appearance to hardware chassis assemblies utilized for desktop computers. The embodiments herein exploit qualitative and quantitative capabilities of IMS analysis to profile targeted polyamines and utilizes hierarchical algorithms to identify COVID-19 positive cases and to differentiate infected test subjects from healthy test subjects.

An embodiment assays biologic markers generated from metabolic pathways and their levels of activity in vivo that are due to viral infection. It features rapid turnaround times (TAT of less than one minute), POC test completion capability, and is characterized by high accuracy, sensitivity, and specificity. The embodiment is designed to operate in non-laboratory locations allowing immediate qualitative diagnostic screening of large numbers of people, allowing laypersons to collect and process test samples safely, comfortably, and cost-effectively.

In one embodiment, an apparatus for detecting a virus may comprise a case comprising a sample inlet configured to receive a sample, the sample inlet comprising a vaporization source configured to vaporize the sample into a plurality of molecules; a dopant holder fluidly coupled to the sample inlet, the dopant holder configured to provide a carrier gas to the sample inlet cavity; a drift tube assembly fluidly coupled to the sample inlet and configured to receive the carrier gas comprising the plurality of molecules, the drift tube assembly comprising: an ionization source positioned at a first end of the drift tube assembly, the ionization source configured to ionize the molecules, an ion gate positioned downstream of the ionization source, the ion gate configured to selectively allow a plurality of molecules of interest to flow towards a second end of the drift tube, and a detector plate positioned at the second end of the drift tube, the detector plate configured to detect the plurality of molecules of interest and collect data on the plurality of molecules of interest; and a processor communicatively coupled to the drift tube, the processor configured to quantify the plurality of molecules of interest and provide a qualitative test result.

The vaporization source may be a tritium radiation source configured to vaporize the sample into the plurality of molecules, the tritium radiation source comprising less than 1 giga-becquerel (GBq) of tritium radiation. The vaporization source may be a heat source configured to vaporize the sample into the plurality of molecules, the heat source comprising a bulb.

The ionization source may comprise a plurality of electrodes, wherein the first end is near an inlet of the drift tube. The plurality of electrodes may comprise a plurality of rings comprising at least one of a plastic, a metal, or a ceramic.

The apparatus may comprise a pressure control assembly configured to provide pressurized air to each of dopant holder and the drift tube assembly; an air pump comprising a motor, the air pump configured to draw air into the apparatus; a moisture trap configured to collect moisture and provide dry air to the pressure control assembly; and a pressure valve in fluid receiving communication with the air pump and fluid providing communication with the moisture trap.

The drift tube assembly may further comprise a drift gas aperture positioned adjacent to the detector plate, the drift tube assembly configured to receive a drift gas from the pressure control assembly.

The apparatus may comprise a display positioned on the outer case and communicatively coupled to the processor, the display configured to receive selections from a user and display the test result.

The apparatus may comprise a sample inlet cover positioned within the sample inlet, the sample inlet cover configured to seal the sample inlet when the apparatus is not in use.

The apparatus may comprise at least one printed circuit board comprising a controller and an amplifier.

In another embodiment, a method for detecting a virus may comprise providing a sample to a sample inlet; selecting a vaporization source, the vaporization source positioned within the sample inlet; vaporizing the sample into a plurality of molecules of interest, the plurality of molecules of interest comprising a plurality of molecules; selecting a carrier gas for binding to and transporting the plurality of molecules; selecting an ion gate pore size of an ion gate positioned within a drift tube assembly based on the size of the of molecules; providing the carrier gas and the polyamines to a first end of the drift tube assembly; ionizing, by an ionization source, the molecules; facilitating the flow of the ionized polyamines through the ion gate to a second end of the drift tube assembly, the second end of the drift tube assembly comprising a detector plate; and detecting, by the detector plate, a quantity of ionized molecules.

The method may comprise storing, the quantity of ionized molecules; and providing a qualitative test result based on the quantity of ionized molecules.

The vaporization source may be a tritium radiation source configured to vaporize the sample into the plurality of molecules, the tritium radiation source comprising less than 1 giga-becquerel (GBq) of tritium radiation. The vaporization source may be a heat source configured to vaporize the sample into the plurality of molecules, the heat source comprising a bulb.

The method may comprise selecting a temperature of the carrier gas, and heating the carrier gas to the selected temperature.

The ionization source may comprise a plurality of ring electrodes comprising at least one of a plastic, a metal, or a ceramic.

In yet another embodiment, a method of sampling a plurality of sample with a virus detecting apparatus may comprise collecting a sample from a subject; adding a reagent to the sample; providing the sample to a sample inlet, the sample inlet positioned on an outer case of the apparatus; running a new test on the sample and storing data collected from the new test in a storage; removing the sample from the sample inlet; and providing, by the apparatus, a qualitative test result.

The method may comprise selecting a vaporization source, the vaporization source positioned within the sample inlet; selecting a carrier gas based on a plurality of molecules of interest of the sample, the carrier gas configured to bind the molecules of interest; and selecting an ion gate pore size of an ion gate positioned within a drift tube assembly based on a size of an ionized structure of the molecules of interest.

The method may comprise vaporizing, by a vaporization source, the sample into a plurality of molecules of interest, the plurality of molecules of interest comprising a plurality of molecules; providing the carrier gas and the molecules of interest to a first end of the drift tube assembly; ionizing, by an ionization source, the molecules of interest; facilitating the flow of the ionized polyamines through the ion gate to a second end of the drift tube assembly, the second end of the drift tube assembly comprising a detector plate; and detecting, by the detector plate, a quantity of ionized molecules.

The vaporization source may be at least one of a tritium radiation source or a heat source; and the ionization source may be a plurality of ring electrodes comprising at least one of a plastic, a metal, or a ceramic.

### BRIEF DESCRIPTION OF THE DRAWINGS

The foregoing and other features of the present disclosure will become more fully apparent from the following description and appended claims taken in conjunction with the accompanying drawings. Understanding that these drawings depict only several implementations in accordance with the disclosure and are therefore not to be considered limiting of its scope, the disclosure will be described with additional specificity and detail through use of the accompanying drawings.

FIG. 1 is a flowchart of a method for determining the presence of viruses, according to an embodiment;

FIG. 2A is a perspective view of an apparatus for determining the presence of viruses, according to an embodiment;

FIG. 2B is a cross-sectional of the apparatus of FIG. 2A, according to an embodiment;

FIG. 2C is a top view of the apparatus of FIG. 2A, according to an embodiment;

FIG. 2D is a back side view of the apparatus of FIG. 2A, according to an embodiment;

FIG. 2E is another back side view of the apparatus of FIG. 2A, according to an embodiment;

FIG. 2F is a perspective view of a sample inlet and a portion of a drift tube assembly of the apparatus of FIG. 2A, according to an embodiment;

FIG. 2G is a perspective view of the drift assembly of FIG. 2E, according to an embodiment;

FIG. 2H is a perspective view of an air pump of the apparatus of FIG. 2A, according to an embodiment;

FIG. 2I is a perspective view of a pressure valve of the apparatus of FIG. 2A, according to an embodiment;

FIG. 2J is a perspective view of a moisture trap of the apparatus of FIG. 2A, according to an embodiment;

FIG. 2K is a perspective side view of the apparatus of FIG. 2A, according to an embodiment;

FIG. 2L is a perspective view of a pressure control assembly of the apparatus of FIG. 2A, according to an embodiment;

FIG. 2M is a top view of a dopant holder of the apparatus of FIG. 2A, according to an embodiment:

FIG. 2N is a perspective view of a computer assembly of the apparatus of FIG. 2A, according to an embodiment;

FIG. 2O is a perspective view of a vent fan of the apparatus of FIG. 2A, according to an embodiment;

FIG. 2P is a perspective view of a solid-state relay of the apparatus of FIG. 2A, according to an embodiment;

FIG. 3 is a flow chart for a method of detecting a virus using an apparatus, according to an embodiment;

FIG. 4 is a flow chart illustrating the sequential flow of the current embodiment of an algorithm for detecting a virus using an apparatus, according to an embodiment;

FIG. 5 is a flow chart illustrating a method for preparing testing equipment for detecting a virus using an apparatus, according to an embodiment;

FIG. 6 is a flow chart illustrating a method of conducting a test for detecting a virus using an apparatus, according to an embodiment;

FIG. 7A illustrates a step of conducting the test for detecting a virus using an apparatus, according to the embodiment of FIG. 6;

FIG. 7B illustrates another step of conducting the test for detecting a virus using an apparatus, according to the embodiment of FIG. 6;

FIG. 7C illustrates another step of conducting the test for detecting a virus using an apparatus, according to the embodiment of FIG. 6;

FIG. 7D illustrates another step of conducting the test for detecting a virus using an apparatus, according to the embodiment of FIG. 6;

FIG. 7E illustrates another step of conducting the test for detecting a virus using an apparatus, according to the embodiment of FIG. 6;

FIG. 7F illustrates another step of conducting the test for detecting a virus using an apparatus, according to the embodiment of FIG. 6;

FIG. 7G illustrates another step of conducting the test for detecting a virus using an apparatus, according to the embodiment of FIG. 6; and

FIG. 7H illustrates another step of conducting the test for detecting a virus using an apparatus, according to the embodiment of FIG. 6.

### DETAILED DESCRIPTION

Reference will now be made to the illustrative embodiments depicted in the drawings, and specific language will be used here to describe the same. It will nevertheless be understood that no limitation of the scope of the claims or this disclosure is thereby intended. Alterations and further modifications of the inventive features illustrated herein, and additional applications of the principles of the subject matter illustrated herein, which would occur to one skilled in the relevant art and having possession of this disclosure, are to be considered within the scope of the subject matter disclosed herein. Other embodiments may be used and/or other changes may be made without departing from the spirit or scope of the present disclosure. The illustrative embodiments described in the detailed description are not meant to be limiting to the subject matter presented.

The embodiments described herein can use a variety of test sample specimen types to determine the presence of viruses, including detection of the SARS-CoV-2 virus that causes COVID-19 and Influenza A and B. Although examples may describe the detection of SARS-CoV-2 or Influenza herein, it is intended that the methods and systems can be used to detect other viruses as well.

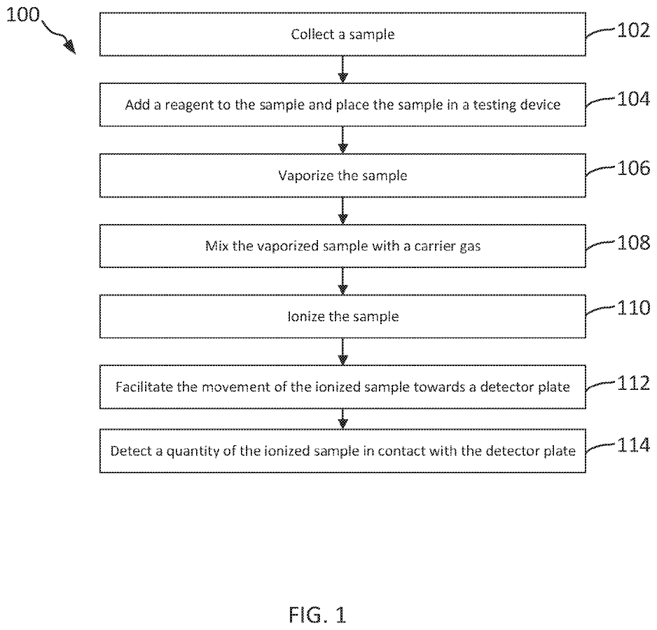

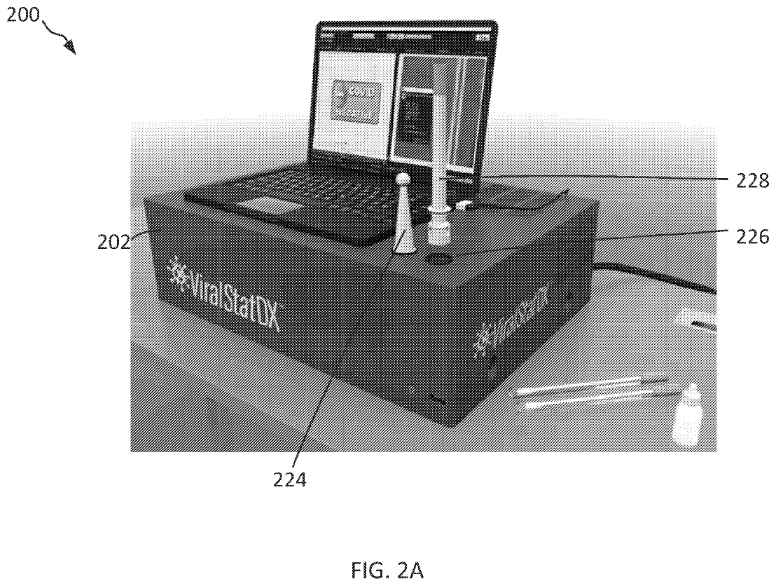

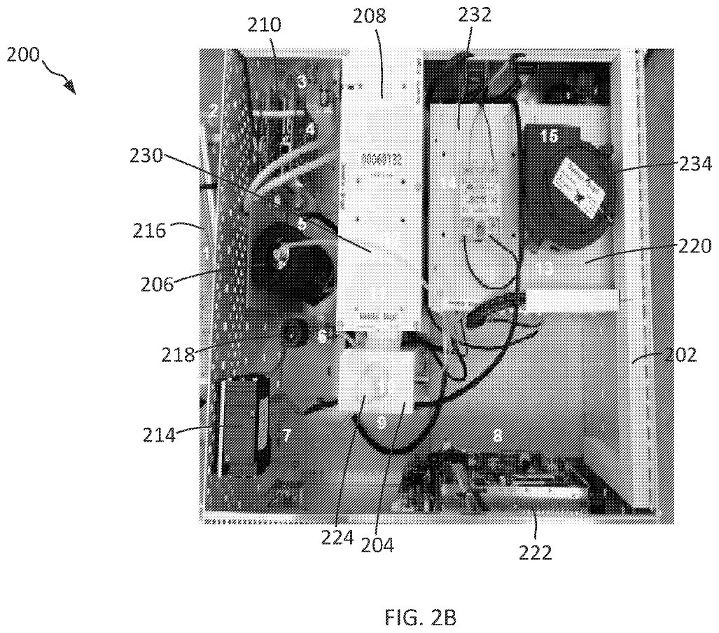

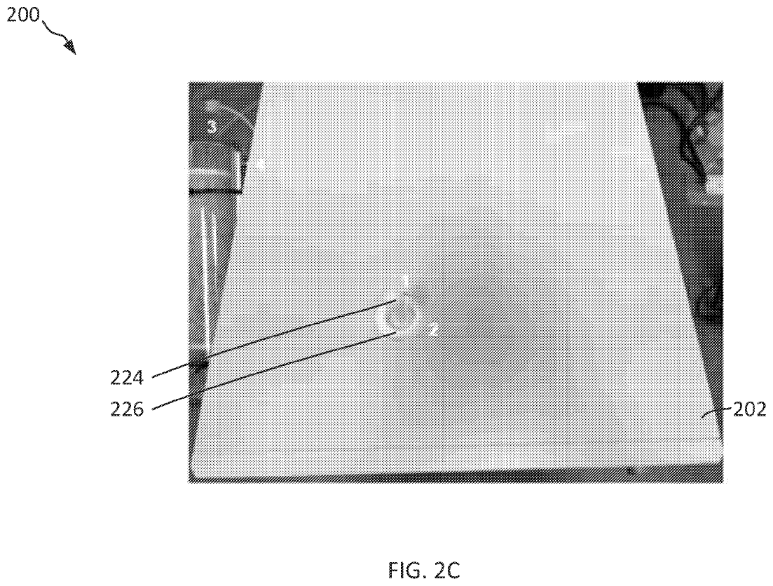

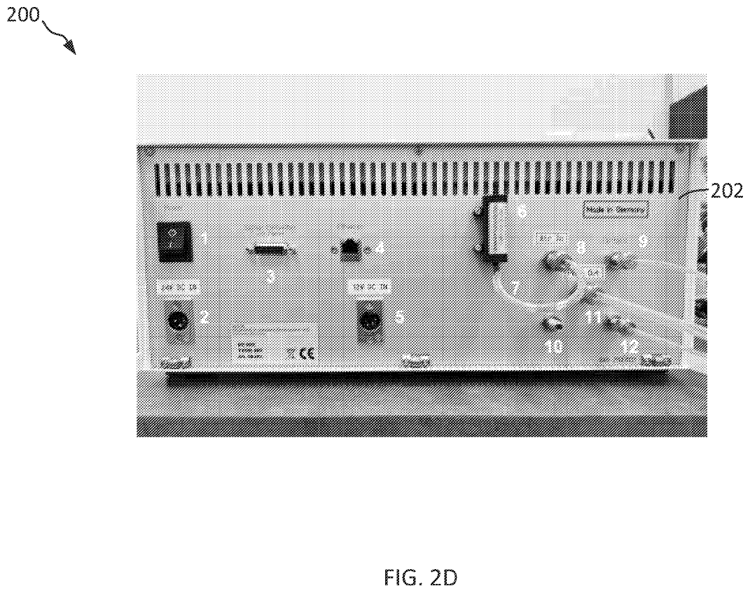

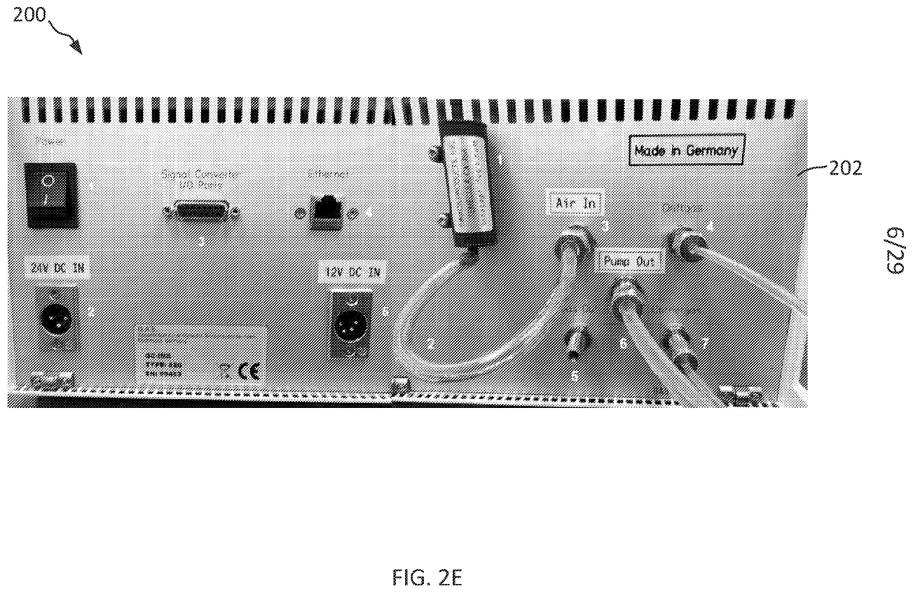

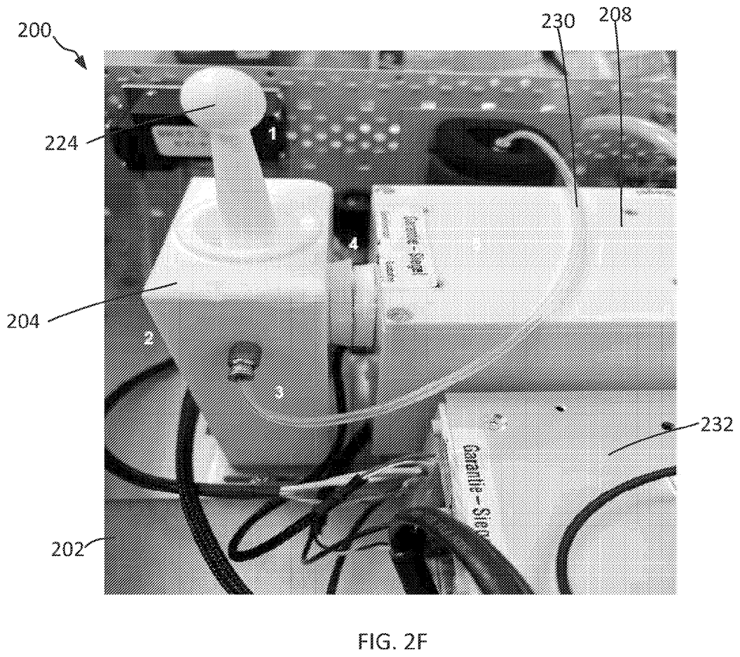

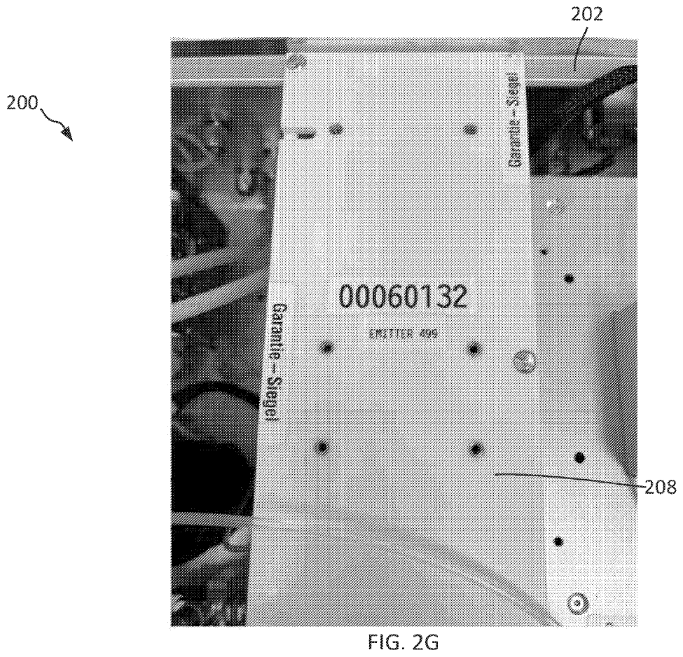

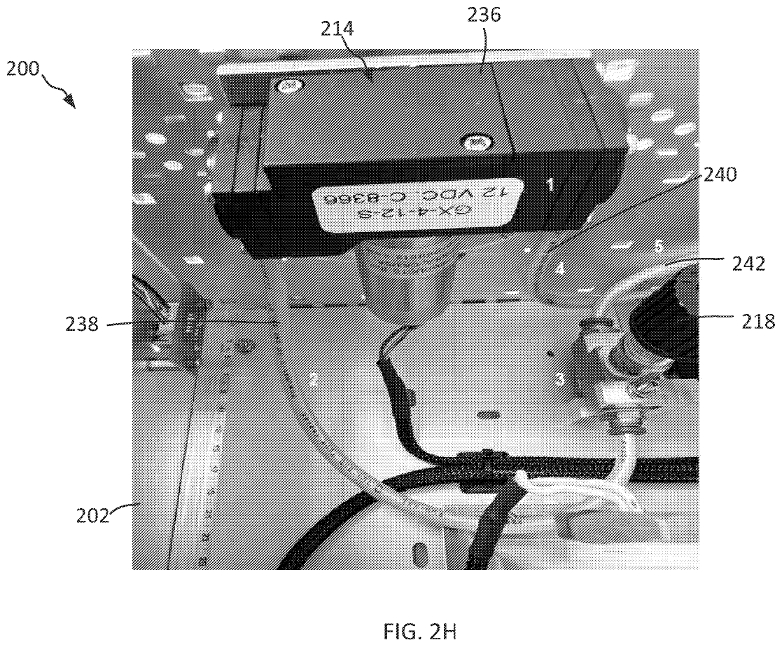

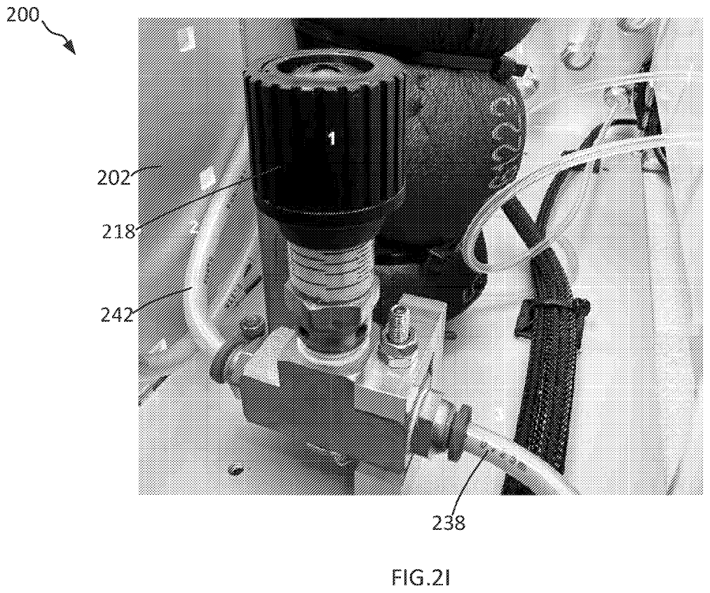

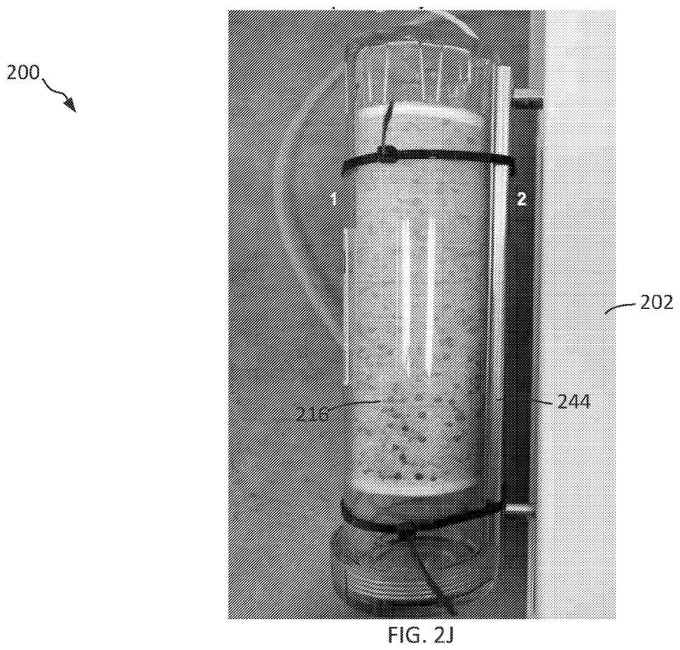

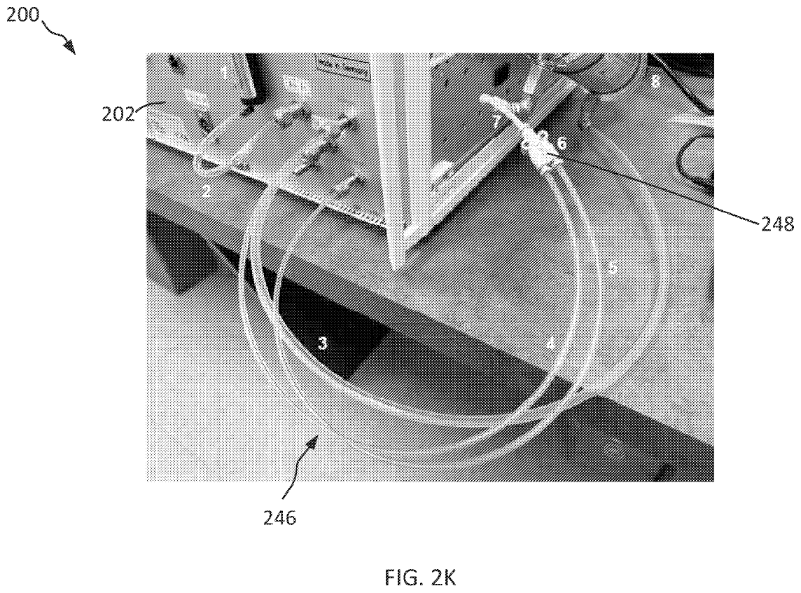

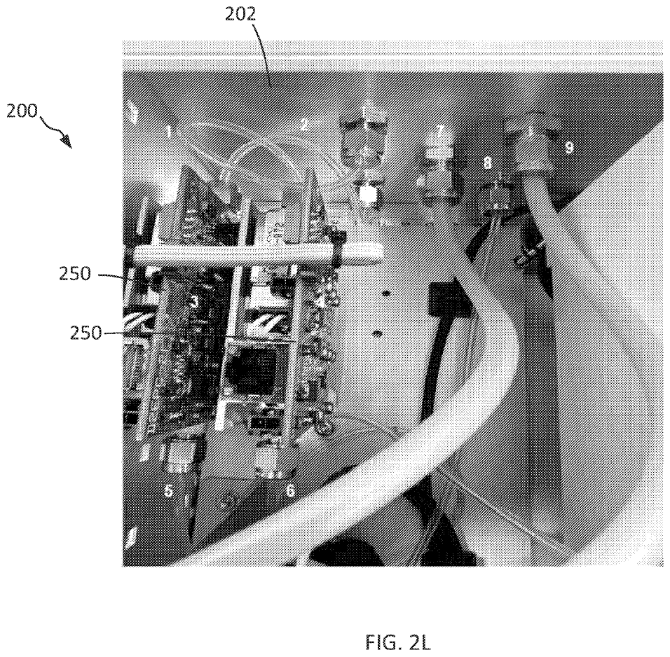

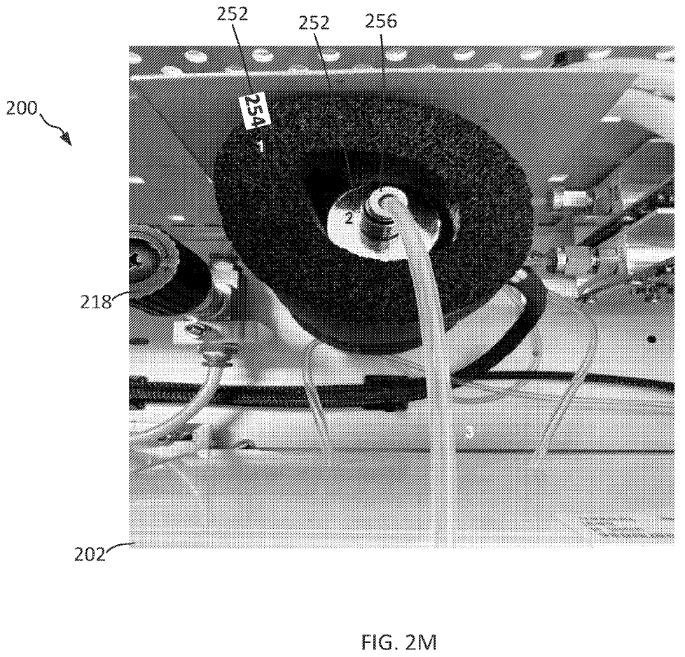

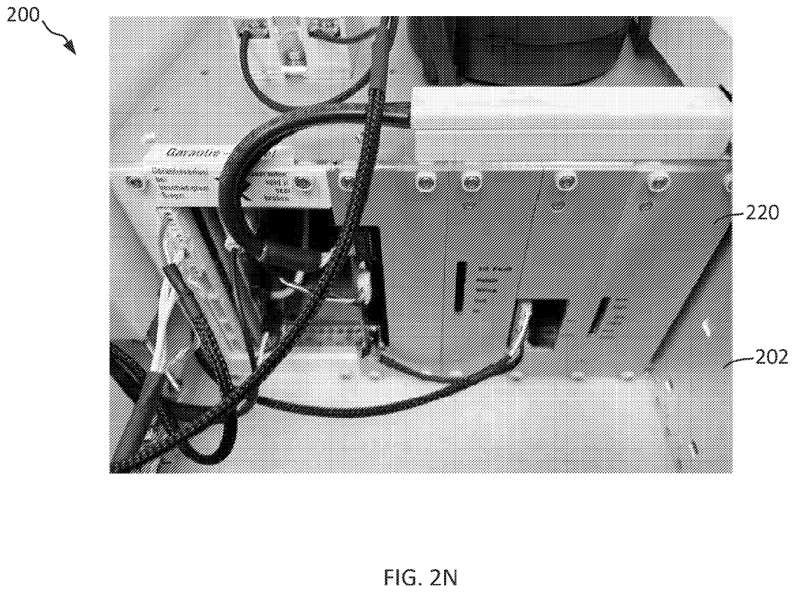

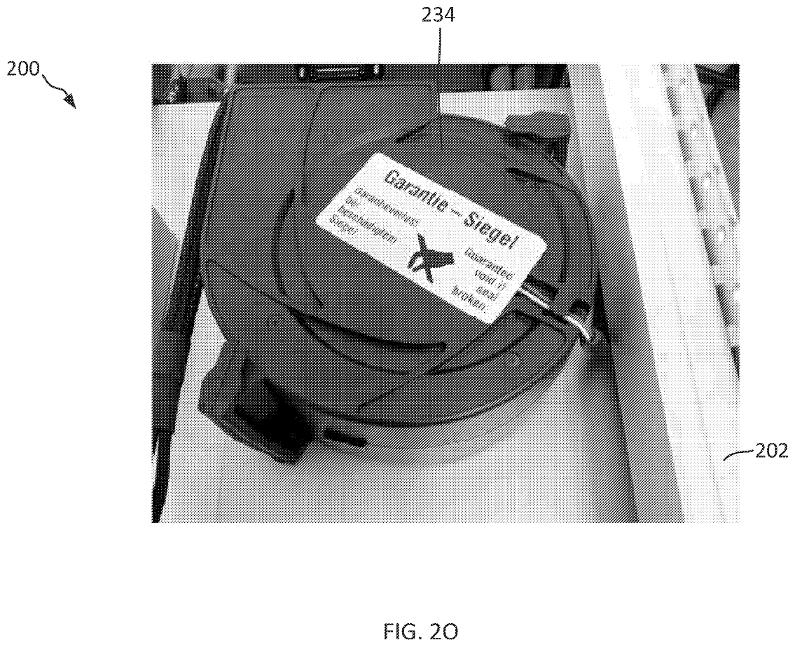

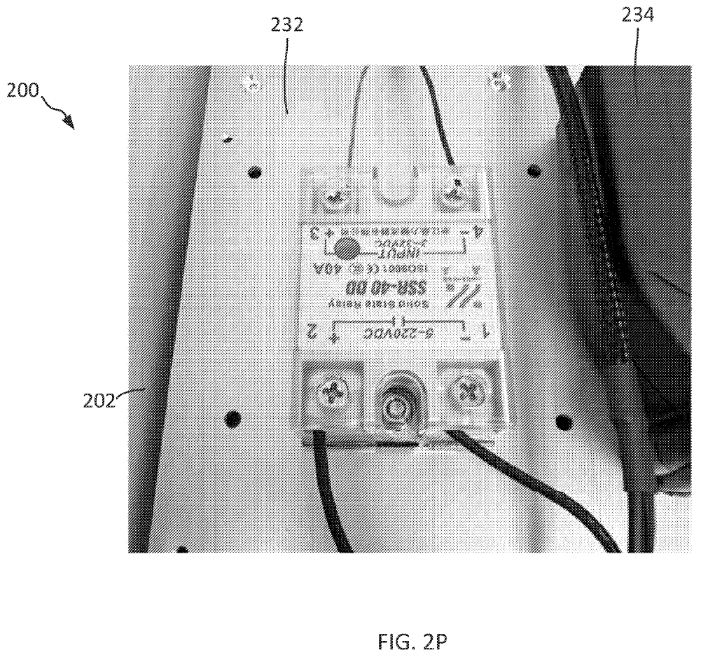

In one configuration, a method 100 for determine the presence of viruses is shown in FIG. 1. The functionalities of the method 100 may be implemented using, or performed by, the components detailed herein in connection with FIGS. 2A-2P. In some embodiments, the apparatus 200 may perform one or more of the functionalities of method 100 together or independently.

In step 102, a sample utilizes saliva collected from under the tongue by a cotton-tipped or synthetic tipped swab. According to this embodiment, in step 104, about one to two drops of 15% KOH (potassium hydroxide) solution can be placed on the swab before placing it in the testing device, allowing for initiation of sample degradation. In step 106, vaporization of the sample occurs. Vaporization of the sample may occur by heating the sample using a heat source such as an incandescent or halogen lamp or other compatible heat source that is contained inside a housing. In other configurations vaporization of same may occur by using a tritium radiation source. The tritium radiation source includes less than 1 giga-becquerel (GBq) of tritium radiation. Next, in step 108, the vaporized sample and a carrier gas are mixed. The carrier gas is configured to bind to the vaporized sample. In some embodiments, the carrier gas is heated to a desired temperature depending on the molecules of interest within he vaporized sample. For example, one can accomplish mixing the sample vapors by sending the heated carrier gas to the sample introduction chamber (e.g., sample inlet cavity, etc.). In step 110, as the mixed vapors (e.g., the vaporized sample and carrier gas mixture, etc.) enter the ionization area of the drift tube assembly, ionization of the sample takes place facilitated by the heat generated by the electrodes. It is here that separate molecules of analytes of interest are converted into ions differing by the shape and size of their chemical structures. The ion gate/shutter within the drift tube assembly is opened and closed for a brief period to allow a swarm of ions into the drift region. In step 112, the various ions then move from a first end of the drift tube assembly near the inlet, towards a detector plate positioned on a second end of the drift tube assembly. Movement of the different ions within the drift tube assembly is dependent on their size and shape as well as their interaction with the incoming drift gas. A constant electrical current is maintained by an array of electrodes throughout the drift tube. Generally, the smaller ions of interest move quickly and reach a detector plate or array positioned on a side of the drift tube assembly opposite the inlet first, followed by larger molecules. In our application, smaller molecules of interest include putrescine, which reaches the detector plate first, followed by molecules of spermidine and also spermine. Each of these analyte molecules differ by one amine group (e.g., two amines in putrescine, three in spermidine, and four in spermine). In step 114, the software records the quantity of each analyte. The relative percentages for each analyte can be obtained by exporting the values into a companion software which does the conversion. The specific algorithm looks for a predetermined value of percentages and determines if a particular analyte exceeds that threshold.

In step 116, a qualitative result is provided based on the quantity of molecules (e.g., analytes, etc.) detected. If only one of the three analytes exceed the threshold value, the sample is considered COVID negative. However, if two or three analytes exceed the threshold values, the sample is considered COVID positive. The result may be displayed on a screen (e.g., LED screen) on the apparatus or communicatively coupled to the apparatus. The result may also be (wired or wirelessly) transmitted to another device, such as a mobile phone or tablet computer, and may be presented as a notification, email message, text message, pop-up message, or the like. The results may be stored in a database.

Various embodiments include a method and an apparatus for the detection of the SARS-CoV-2 virus and its variants that cause COVID-19 using free polyamines from a human sublingual saliva sample, utilizing targeted ion mobility spectrometry, incorporating the following elements.

An apparatus 200 for detecting a virus is shown in FIGS. 2A-2P. According to this embodiment, the apparatus 200 includes an outer metal case 202 (or other rigid housing). The outer metal case 202 is configured to house various components of the apparatus 200 and configured to couple various exterior components to the apparatus 200.

The apparatus 200 also includes a sample inlet 204. The sample inlet 204 is configured to receive a sample. For example, the sample may be a swab containing saliva. The sample inlet 204 defines a sample inlet cavity (e.g., sample introduction cavity, etc.). The sample inlet cavity is configured to vaporize (e.g., volatize, etc.) the sample. For example, a heat source or a tritium radiation source may be positioned within the sample inlet cavity to vaporize the sample.

The apparatus 200 further includes a dopant holder 206. The dopant holder 206 is configured to house a dopant or a carrier gas, to provide to the sample inlet chamber. For example, the sample inlet chamber receives the carrier gas and mixes the carrier gas with the vaporized sample.

The apparatus 200 also includes a drift tube assembly 208. The drift tube assembly 208 includes an inlet fluidly coupled to the sample inlet cavity. The inlet includes an ionization source. For example, the ionization source may a plurality of electrodes such as rings made of a plastic, a metal, or a ceramic. In some embodiments, electrodes are placed through the drift tube assembly 208 to provide a drift current within the drift tube assembly 208. The drift tube assembly 208 includes an ion gate of a pore size that allows the molecules or analytes to selectively pass through the ion gate. Downstream of the ion gate, the drift tube assembly 208 includes a detector plate. The detector plate is configured to detect a quantity of molecules. For example, the carrier gas sample mixture is provided to the inlet of the drift tube assembly and ionized. The electrodes facilitate flow through the ion gate towards the detector plate. The detector plate then quantifies the number of molecules that have reached the detector plate.

The apparatus 200 further includes a pressure control assembly 210. The pressure control assembly 210 is configured to provide pressurized air to each of the dopant holder and the drift tube assembly. The pressure control assembly 210 may include various components such as a filter. The drift tube assembly 208 may also include a gas aperture near the detector plate. The drift tube assembly is configured to receive drift gas from the pressure control assembly 210.

The apparatus 200 also includes an air pump 214 and a moisture trap 216. The air pump 214 is configured to draw air into the apparatus 200 and the moisture trap is configured to collect moisture and provide dry air to the pressure control assembly 210. The apparatus 200 further includes a pressure valve 218. The pressure valve 218 is in fluid receiving communication with the air pump 214 and in fluid providing communication with the moisture trap 216.

FIG. 2A illustrates an apparatus with test collection components and is communicatively coupled to a notebook computer. The computer may be a laptop computer, desktop computer, tablet computer, mobile phone, or other computing device. The computer may be a separate component or the computer may be integrated into the apparatus, such that the apparatus includes a processor, a non-transitory computer-readable memory containing instructions to be executed by the computer and containing data (e.g., in the Access Database), and a display (e.g., LED).

The apparatus 200 may include a processor 220 (e.g., a computer readable medium, a motherboard, etc.) configured to receive, analyze, and store data, such as the quantity of molecules, detected by the detector plate. For example, the computer may analyze the data and provide a qualitative result (e.g., positive result, negative result, etc.) to the user. The apparatus also includes a display 222 (e.g., LCD display, etc.) configured to display (e.g., provide, show, etc.) data, results, or other information to the user. The display may also be configured to receive selections of various settings from the user.

The apparatus may include a sample inlet cover 224 placed in the sample inlet 204. The sample inlet cover 204 is configured to seal the sample inlet cavity and prevent unwanted objects or dust from entering the sample inlet cavity.

As shown in FIGS. 2A to 2G and 2N to 2P, the outer case 202 includes an opening 226. The opening 226 is configured to receive a test kit insertion tube 228. When the apparatus 200 is not in use, the sample inlet cover 224 us placed in the opening 226.

The apparatus also includes a plurality of tubing 230 configured to receive a dopant from the dopant holder 206 and provide the dopant to the sample inlet 204. The apparatus 200 also includes a solid-state relay 232 and a vent fan 234.

As shown in FIGS. 2H and 2I, the air pump 214 includes a motor 236, a first air tube 238 configured to receive air from the pump and provide air to the pressure release valve 218, a second air tube 240 configured to receive air from the air intake and provide air to the air pump 214. The apparatus 200 also includes a third air tube 242 configured to receive air from the pressure release valve 218 and provide air to the moisture trap 216.

As shown in FIG. 2J, the moisture trap 216 may positioned on an exterior portion of the outer case 202. For example, the apparatus 200 may include a bracket 244 attaching the moisture trap 216 to the outer case 202.

As shown in FIG. 2K, the apparatus 200 may also include a plurality of exterior tubing 246 and a splitter 248. The plurality of tubing is fluidly coupled to the moisture trap 216. The splitter 248 splits the flow from the moisture trap 216 into at least two separate flow paths to provide a fluid (e.g., air, dry air, etc.) to various components within the outer casing 202. For example, the plurality of exterior tubing 246 may provide dry air to the pressure control assembly 210 for the carrier gas and for drift gas.

As shown in FIG. 2L, the pressure control assembly 210 includes a plurality of circuit boards (e.g., EPC boards, etc.) 250.

As shown in FIG. 2M, the dopant assembly 206 includes a dopant holder 252 configured to house a dopant, a heating jacket 254 configured to insulate the dopant holder 252, and an inlet 256 configured to receive dry air from the moisture trap 216.

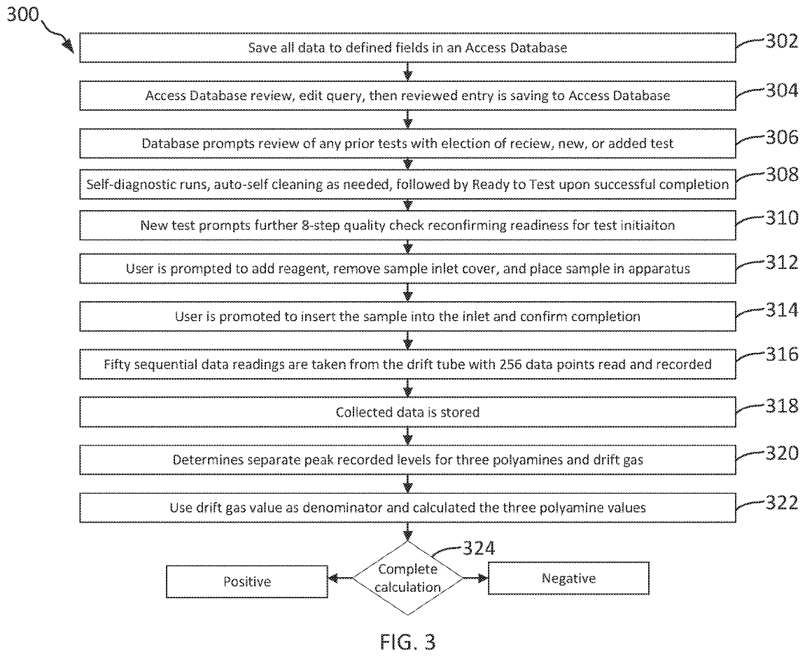

In one configuration, a method 300 for detecting viruses is shown in FIG. 3. The functionalities of the method 200 may be implemented using, or performed by, the components detailed herein in connection with FIGS. 2A-2P. In some embodiments, the apparatus 200 may perform one or more of the functionalities of method 300 together or independently.

FIG. 3 presents a summary logic diagram illustrating the sequential flow of the current embodiment of an algorithm. The functionalities of the method 300 may be implemented using, or performed by, the components detailed herein in connection with FIGS. 2A-2P. In some embodiments, the apparatus 200 may perform one or more of the functionalities of the method 300 together or independently.

In step 302, all data is saved to defined fields in an Access Database, which may be stored internally or externally to the apparatus 200. In step 304, the Access Database is reviewed, a query may be edited, and then the reviewed entry is saved to the Access Database. In step 306, the Access Database prompts a review of any prior tests with the election of review, new, or added test. In step 308, the self-diagnostic runs, auto-self-cleaning occurs as needed, and is followed by the prompt “Ready to Test” upon successful completion. In step 310, a selection of a new test prompts a further 8-step quality check reconfirming readiness for test initiation. In step 312, the user is prompted to add reagent, remove the sample inlet cover, and place the sample in the apparatus. In step 314, the user is prompted to insert the sample into the inlet and confirm completion. In step 316, fifty sequential data readings are taken from the drift tube with 256 data points read and recorded. In step 318, the collected data is stored in a storage. In step 320, separate peak recorded levels for three polyamines and drift gas are determined. In step 322, the three polyamine values are calculated with the drift gas value being used as the denominator. In step 324, the calculation is completed. If two or more polyamine thresholds are met then a result of POSITIVE (e.g., covid positive), if 0-1 polyamine thresholds are met then a result of NEGATIVE (e.g., covid negative).

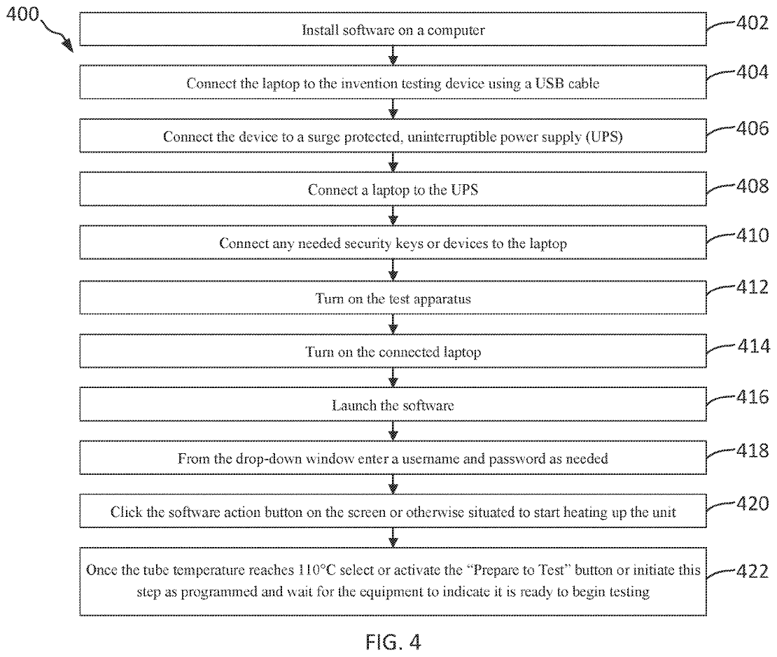

In order to conduct diagnostic testing, a method for preparing the testing equipment is as follows and shown in FIG. 4. The functionalities of the method 400 may be implemented using, or performed by, the components detailed herein in connection with FIGS. 2A-2P. In some embodiments, the apparatus 200 may perform one or more of the functionalities of the method 400 together or independently.

In step 402, software may be installed on a computer, which may be communicatively coupled to the apparatus 200 or integrated into apparatus 200. In step 404, the computer is connected to the testing device (e.g., apparatus 200) using a USB cable. In step 406, the device is connected to a surge protected, uninterruptible power supply (UPS). In step 408, the laptop is connected to the UPS. In step 410, the any necessary security keys or devices are connected to the laptop. In step 412, the test machine is turned on. In step 414, the connected laptop is tuned on. In step 416, the software is launched. In step 418, a username and password are entered as needed from a drop-down menu. In step 420, the software action button on the screen is selected (e.g., clicked, etc.) to begin heating up the unit. In step 422, once the tube temperature reaches 110 °C, the “Prepare to Test” button is selected (e.g., activated, etc.), and then the equipment indicates it is ready to begin testing.

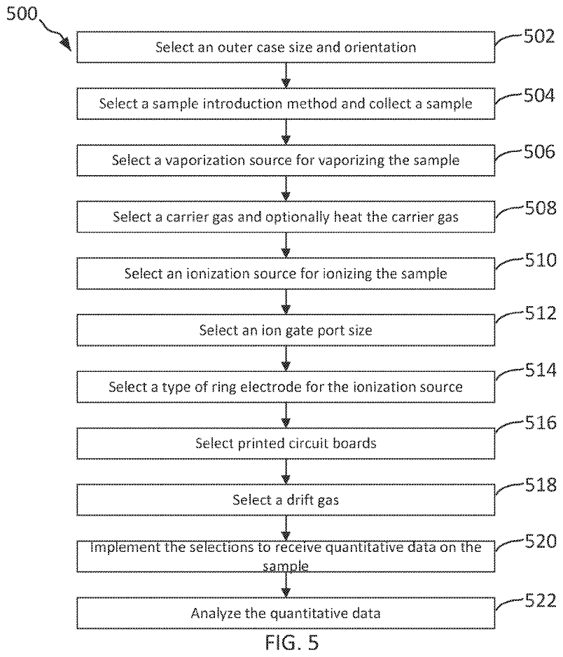

As shown in FIG. 5 and described below is method 500 for detecting a virus using an apparatus, such as the apparatus 200. For example, the method 500 may include one or more of the following steps. The functionalities of the method 500 may be implemented using, or performed by, the components detailed herein in connection with FIGS. 2A-2P. In some embodiments, the apparatus 200 may perform one or more of the functionalities of the method 500 together or independently.

In step 502, the architecture and dimension of the outer metal case is selected. The user may select a size and orientation of the architecture of the apparatus (e.g., the orientation of the outer metal case). Step 502 is a prerequisite for making sure that all the components of the device will fit nicely inside the allotted space, whether in the preferred horizontal form factor or in a vertical form factor.

In step 504, the introduction mechanism is selected, the sample is collected (e.g., liquid held in an absorbent swab, etc.), and is then inserted (e.g., placed, etc.) into the testing device/machine. For example, if the sample is to be collected using a swab, the sample is extracted onto the introduction mechanism and then the introduction mechanism is inserted into the testing device/machine. Step 504 may also include providing a reagent to the sample and placing the sample in the sample inlet.

In step 506, the volatilization source (e.g., heat source, radiation source, etc.) used for volatilizing/vaporizing the sample (e.g., a 50W to 100W bulb) is selected. For example, the user may select to use a heat source, such as a bulb, or a tritium radiation source.

In step 508, the selection of the carrier gas is made and is dependent on the type of volatized molecules (e.g., polyamines, analytes, etc.) under consideration. Additionally, whether a single or a mixture of gases are used as a carrier gas is also dependent on the chemical structures of the molecules under consideration. Further, in step 508, depending on the molecules of interest within the sample, the user may select a carrier gas and heat the carrier gas to be within a specific temperature range.

In step 510, the ionization source is selected. For example, the ionization source mat be electrodes placed at the beginning of the drift tube which consists of a series of plastic, metal, or ceramic electrodes.

In step 512, the ion gate pore size of the drift tube assembly is selected and is dependent on the chemical structure of the ionized analytes under consideration. Bigger ions require larger pores and smaller ions require smaller pore sizes. In step 512, the ion gate is selected such that even the largest ion will pass through the pores of the ion gate.

In step 514, select the type of ring electrodes (e.g., plastic electrodes, ceramic electrodes, metal electrodes, etc.) of the ionization source within the analyzer/drift tube. For example, the ionization source may include 21 rings and be composed of a plastic, a metal, or a ceramic, though other amounts of rings and materials may be utilized that are consistent with this disclosure. The rings may be spaced a distance apart within the drift tube assembly, such that the electrodes facilitate the flow of the ionized sample towards the detector plate.

In step 516, an appropriate pneumatic pump and filtering devices are selected, which are connected to the sample introduction chamber, drift tube, control valves, and exhaust through tubing.

In step 518, printed circuit boards (PCBs) including controller, high voltage, and amplifier board capabilities with components specified for the testing device are selected and may be placed inside the outer case and are appropriately connected to other components utilizing fasteners and cables.

In step 520, a drift gas is selected. The drift gas is provided to the drift tube assembly. According to some embodiments, the drift gas may be air.

In step 522, settings on the device are implemented so that the saliva sample being tested is properly vaporized and polyamines of interest are correctly ionized and accurately quantified. The processor utilizes algorithms to analyze the quantitative data generated in order to determine a positive or negative qualitative diagnostic result. The positive or negative result is communicated (e.g., displayed via an LCD screen, etc.) to the user.

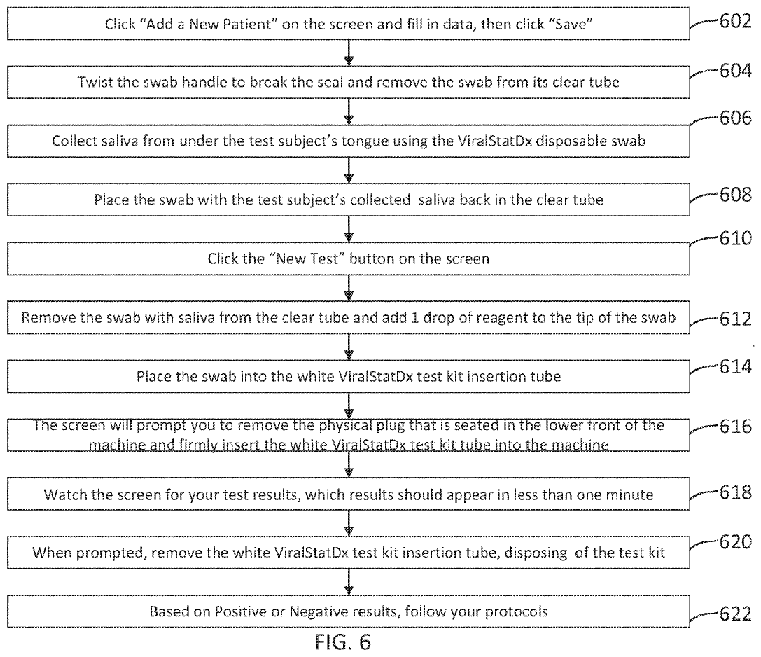

As shown in FIG. 6, a method 600 of conducting a test is illustrated. The method 600 may include one or more of the following steps. The functionalities of the method 600 may be implemented using, or performed by, the components detailed herein in connection with FIGS. 2A-2P. In some embodiments, the apparatus 200 may perform one or more of the functionalities of the method 600 together or independently.

In step 602, the user clicks “Add a New Patient” on the screen and fills in data, then clicks “Save.” In step 604, the user twists the swab handle to break the seal and removes the swab from its clear tube. In step 606, the user collects saliva from under the test subject's tongue using the disposable swab. In step 608, the user places the swab with the test subject's collected saliva back in the clear tube. In step 610, the user clicks the “New Test” button on the screen. In step 612, the user removes the swab with saliva from the clear tube and adds about 1 drop of reagent to the tip of the swab. In step 614, the user places the swab into the white test kit insertion tube. In step 616, the user is prompted, by the screen, to remove the physical plug that is seated in the lower front of the machine and firmly insert the white test kit tube into the machine. In step 618, the test results appear on the screen, which results should appear in less than one minute. In step 620, the user, when prompted to on the screen, removes the white test kit insertion tube, and disposes of all parts of the test kit using a suitable biohazard container. The user then replaces the machine's physical plug into the front of the machine. In step 622, based on a positive or negative result, the user may be instructed to follow a certain protocol.

Additionally, the user may repeat steps to continue testing other samples. Once the user is done testing samples, the user turns off the system.

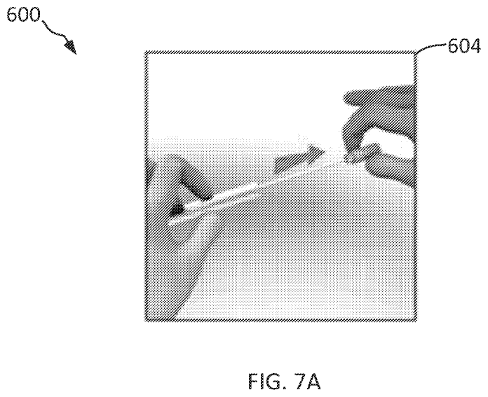

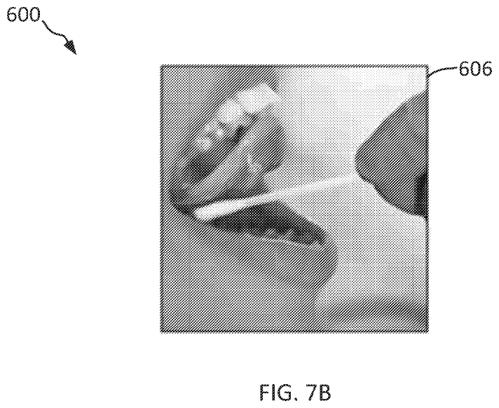

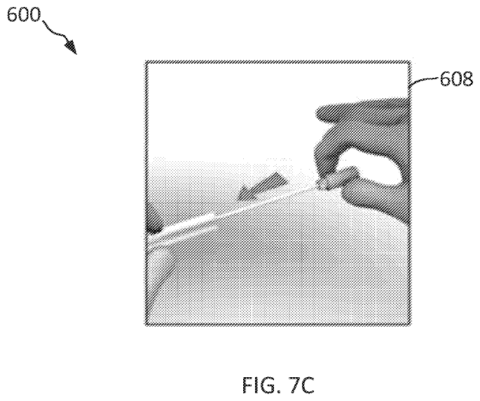

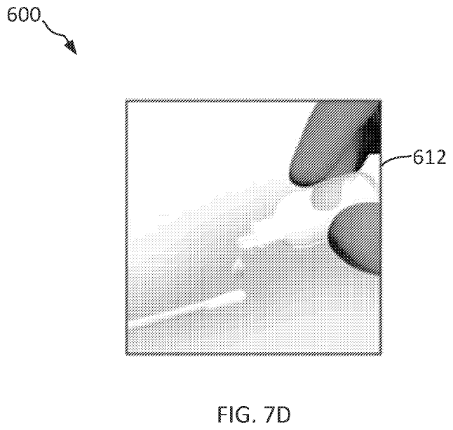

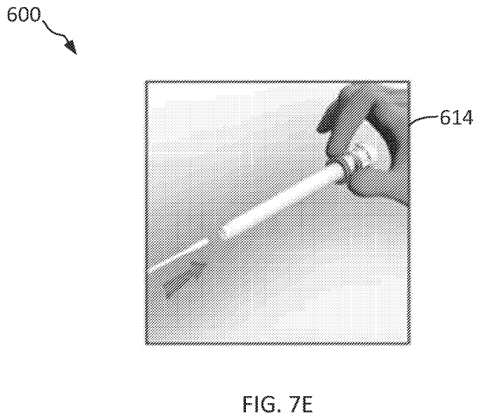

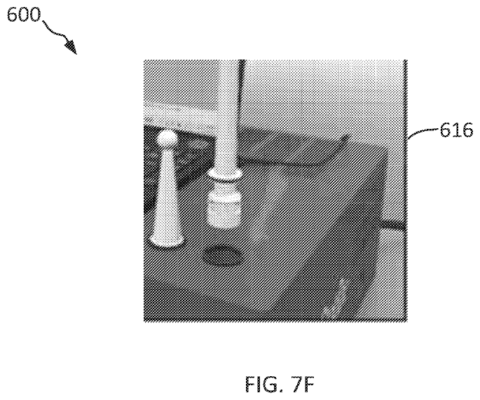

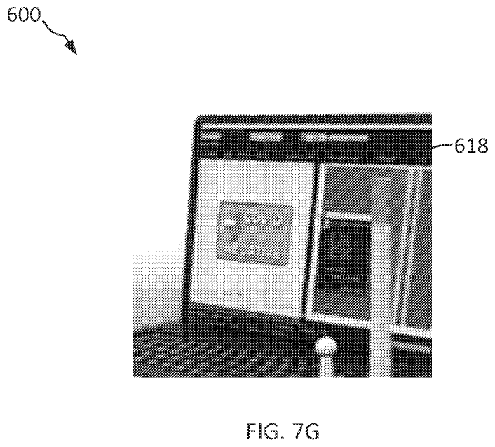

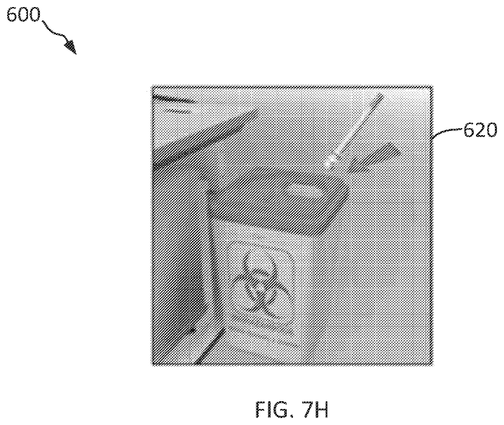

FIGS. 7A-7H illustrate a method for preparing the testing equipment along with the method described in FIG. 6. FIG. 7A illustrates a user twisting the swab handle to break the seal and removing the swab from its clear tube.

FIG. 7B illustrates the user collecting saliva from under the test subject's tongue using the disposable swab.

FIG. 7C illustrates the user placing the swab with the test subject's collected saliva back into the clear tube.

FIG. 7D illustrates the user removing the swab with saliva from the clear tube and adding about 1 drop of reagent to the tip of the swab.

FIG. 7E illustrates the user placing the swab into the white test kit insertion tube.

FIG. 7F illustrates the user firmly inserting the white test kit tube into the machine.

FIG. 7G illustrates the test results appearing on the screen connecting to the apparatus.

FIG. 7H illustrates the user disposing of the sample in a proper disposal bin once testing is complete.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of this disclosure or the claims.

Embodiments implemented in computer software may be implemented in software, firmware, middleware, microcode, hardware description languages, or any combination thereof. A code segment or a machine-executable instruction may represent a procedure, function, subprogram, program, routine, subroutine, module, software package, class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code, it being understood that software and control hardware can be designed to implement the systems and methods based on the description herein.

When implemented in software, the functions may be stored as one or more instructions or code on a non-transitory, computer-readable, or processor-readable storage medium. The steps of a method or algorithm disclosed herein may be embodied in a processor-executable software module, which may reside on a computer-readable or processor-readable storage medium. A non-transitory computer-readable or processor-readable media includes both computer storage media and tangible storage media that facilitates the transfer of a computer program from one place to another. A non-transitory, processor-readable storage media may be any available media that can be accessed by a computer. By way of example, and not limitation, such non-transitory, processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that can be accessed by a computer or processor. Disk and disc, as used herein, include compact disc (CD), laser disc, optical disc, digital versatile disc (DVD), Blu-ray disc, and floppy disk, where “disks” usually reproduce data magnetically, while “discs” reproduce data optically with lasers. Combinations of the above should also be included within the scope of computer-readable media. Additionally, the operations of a method or algorithm may reside as one or any combination or set of codes and/or instructions on a non-transitory, processor-readable medium and/or computer-readable medium, which may be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the principles defined herein may be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What is claimed is:

1. An apparatus for detecting a virus comprising:
a case comprising a sample inlet configured to receive a sample, the sample inlet comprising a vaporization source configured to vaporize the sample into a plurality of molecules;
a dopant holder fluidly coupled to the sample inlet, the dopant holder configured to provide a carrier gas to the sample inlet cavity;
a drift tube assembly fluidly coupled to the sample inlet and configured to receive the carrier gas comprising the plurality of molecules, the drift tube assembly comprising:
an ionization source positioned at a first end of the drift tube assembly, the ionization source configured to ionize the molecules,
an ion gate positioned downstream of the ionization source, the ion gate configured to selectively allow a plurality of molecules of interest to flow towards a second end of the drift tube, and
a detector plate positioned at the second end of the drift tube, the detector plate configured to detect the plurality of molecules of interest and collect data on the plurality of molecules of interest; and
a processor communicatively coupled to the drift tube, the processor configured to quantify the plurality of molecules of interest and provide a qualitative test result.

2. The apparatus of claim 1, wherein vaporization source is a tritium radiation source configured to vaporize the sample into the plurality of molecules, the tritium radiation source comprising less than 1 giga-becquerel (GBq) of tritium radiation.

3. The apparatus of claim 1, wherein the vaporization source is a heat source configured to vaporize the sample into the plurality of molecules, the heat source comprising a bulb.

4. The apparatus of claim 1, wherein the ionization source comprises a plurality of electrodes, wherein the first end is near an inlet of the drift tube.

5. The apparatus of claim 4, wherein the plurality of electrodes comprise a plurality of rings comprising at least one of a plastic, a metal, or a ceramic.

6. The apparatus of claim 1, further comprising:
a pressure control assembly configured to provide pressurized air to each of dopant holder and the drift tube assembly;
an air pump comprising a motor, the air pump configured to draw air into the apparatus;
a moisture trap configured to collect moisture and provide dry air to the pressure control assembly; and
a pressure valve in fluid receiving communication with the air pump and fluid providing communication with the moisture trap.

7. The apparatus of claim 6, wherein the drift tube assembly further comprises a drift gas aperture positioned adjacent to the detector plate, the drift tube assembly configured to receive a drift gas from the pressure control assembly.

8. The apparatus of claim 1, further comprising a display positioned on the outer case and communicatively coupled to the processor, the display configured to receive selections from a user and display the test result.

9. The apparatus of claim 1, further comprising a sample inlet cover positioned within the sample inlet, the sample inlet cover configured to seal the sample inlet when the apparatus is not in use.

10. The apparatus of claim 1, further comprising at least one printed circuit board comprising a controller and an amplifier.

11. A method for detecting a virus comprising:
providing a sample to a sample inlet;
selecting a vaporization source, the vaporization source positioned within the sample inlet;
vaporizing the sample into a plurality of molecules of interest, the plurality of molecules of interest comprising a plurality of molecules;
selecting a carrier gas for binding to and transporting the plurality of molecules;
selecting an ion gate pore size of an ion gate positioned within a drift tube assembly based on the size of the of molecules;
providing the carrier gas and the polyamines to a first end of the drift tube assembly;
ionizing, by an ionization source, the molecules;
facilitating the flow of the ionized polyamines through the ion gate to a second end of the drift tube assembly, the second end of the drift tube assembly comprising a detector plate; and
detecting, by the detector plate, a quantity of ionized molecules.

12. The method of claim 11, further comprising:
storing, the quantity of ionized molecules; and
providing a qualitative test result based on the quantity of ionized molecules.

13. The method of claim 11, wherein the vaporization source is a tritium radiation source configured to vaporize the sample into the plurality of molecules, the tritium radiation source comprising less than 1 giga-becquerel (GBq) of tritium radiation.

14. The method of claim 11, wherein the vaporization source is a heat source configured to vaporize the sample into the plurality of molecules, the heat source comprising a bulb.

15. The method of claim 11, further comprising selecting a temperature of the carrier gas, and heating the carrier gas to the selected temperature.

16. The method of claim 11, wherein the ionization source comprises a plurality of ring electrodes comprising at least one of a plastic, a metal, or a ceramic.

17. A method of sampling a plurality of sample with a virus detecting apparatus, the method comprising:
collecting a sample from a subject;
adding a reagent to the sample;
providing the sample to a sample inlet, the sample inlet positioned on an outer case of the apparatus;
running a new test on the sample and storing data collected from the new test in a storage;
removing the sample from the sample inlet; and
providing, by the apparatus, a qualitative test result.

18. The method of claim 17 further comprising:
selecting a vaporization source, the vaporization source positioned within the sample inlet;
selecting a carrier gas based on a plurality of molecules of interest of the sample, the carrier gas configured to bind the molecules of interest; and
selecting an ion gate pore size of an ion gate positioned within a drift tube assembly based on a size of an ionized structure of the molecules of interest.

19. The method of claim 18, further comprising:
vaporizing, by a vaporization source, the sample into a plurality of molecules of interest, the plurality of molecules of interest comprising a plurality of molecules;
providing the carrier gas and the molecules of interest to a first end of the drift tube assembly;
ionizing, by an ionization source, the molecules of interest;
facilitating the flow of the ionized polyamines through the ion gate to a second end of the drift tube assembly, the second end of the drift tube assembly comprising a detector plate; and
detecting, by the detector plate, a quantity of ionized molecules.

20. The method of claim 19, wherein:
the vaporization source is at least one of a tritium radiation source or a heat source; and
the ionization source is a plurality of ring electrodes comprising at least one of a plastic, a metal, or a ceramic.
